(19) United States
(12) Patent Application Publication
(10) Pub. No.: US 2005/0209651 A1
(43) Pub. Date: Sep. 22, 2005

(54) System and method for treatment of sexual dysfunction

(75) Inventors: Tracy Cameron, Dallas, TX (US); John H. Erickson, Plano, TX (US)

Correspondence Address: FULBRIGHT & JAWORSKI, L.L.P. (ANS), 2200 ROSS AVENUE, SUITE 2800, DALLAS, TX, 75201, US

(73) Assignee: Advanced Neuromodulation Systems, Inc., Pano, TX (US)

(21) Appl. No.: 11/078,910

(22) Filed: Mar. 11, 2005

Related U.S. Application Data
(63) Continuation of application No. 10/283,620, filed on Oct. 30, 2002.

(51) Int. Cl. A61N 1/18

(52) U.S. Cl. 607/39

(57) ABSTRACT

The invention relates to a method of treating sexual dysfunction. The method includes stimulating sacral nerve roots, S2 and/or S3, with electromagnetic stimulation in or about the sacrum. The stimulation may be achieved with leads terminating in electrodes that extend with an emerging sacral nerve root or extend transverse the sacrum at or about the location of the nerve root to be stimulated. Alternately, spiraled electrodes and nerve cuffs may be used to stimulate the nerve root within the sacrum, as it emerges from the sacrum, or outside the sacrum. In this manner, the selected sacral nerve root may be stimulated to provide genital stimulation and thereby induce orgasm.

### CROSS-REFERENCE TO RELATED APPLICATIONS

The present application is a continuation of co-pending, commonly assigned, U. S. patent application Ser. No. 10/283,620 entitled “SYSTEM AND METHOD FOR TREATMENT OF SEXUAL DYSFUNCTION,” filed Oct. 30,2002, the disclosure of which are hereby incorporated herein by reference.

### TECHNICAL FIELD

The present invention, in general, relates to treatment of sexual dysfunction. More specifically, the invention relates to genital stimulation through electromagnetic stimulation of sacral nerve roots, effectively inducing orgasm.

### BACKGROUND OF THE INVENTION

A study published in the Journal of the American Medical Association reported 43% of women and 31% of men are considered to have sexual dysfunction. Of the 43% of women, 22% had low sexual desire, 14% had problems with arousal, and 7% experienced pain during intercourse. A number of pharmacological and psychological treatments have been developed to treat this disorder. However, there remains a group of patients refractory to current methods.

Spinal cord stimulation for treating orgasmic dysfunction has been described in U.S. Patent Meloy et al. (U.S. Pat. No. 6,169,924). Stimulating electrodes are placed in the spinal canal parallel with the spinal cord. The spinal cord is then stimulated with electric signals inducing genital stimulation and possibly orgasm.

The region of the spinal cord typically associated with genital dermatomes is given the denotation of S3. This denotation is derived from the fact that nerve roots extending from this region of the spinal cord exit the spinal column in the region of the third sacral vertebra. The S3 region of the spinal cord is generally located about the T11 through L3 vertebrae. The spinal cord generally terminates above the L4 vertebra. Meloy et al. (U.S. Pat. No. 6,169,924) teaches the stimulation of the spinal cord with electrodes inserted between the T11 and L3 vertebrae.

However, direct stimulation of the spinal cord has several disadvantages for treating this dysfunction. First, the spinal cord is not fixed in place. As such, it moves as patients alter their position or posture. Additionally, the region of the spinal cord associated with the genital region is small and entangled with other nerve fibers. Thus, movement of the nerve bundle relative to the stimulating electrodes would likely result in a failure to stimulate the desired nerve bundle and possibly stimulating an undesired nerve bundle.

Another problem associated with stimulation of the spinal cord is tied to the location of those nerves associated with genital stimulation. Genital stimulation is associated with nerves near the base or end of the spinal cord. In this region, the spinal cord tapers, spawning a significant number of nerve roots in a very small region. For this reason, it is difficult to locate those nerves that will lead to genital stimulation and induce orgasm.

Brindley stimulation of nerve roots has been used in the control of bladder and sphincter finction in patients with spinal injuries by stimulating the appropriate sacral nerve roots with interrupted electric pulses has been used to restore bladder function and bowel function in these patients. Typically, Brindley stimulation is the stimulation of motor neurons. Often, the sensory neurons are severed in the surgery. With the severing of the sensory neurons, these patients cannot feel sensations in the affected dermatomes. As a side effect, these stimulators have also been shown to induce penile erection in male patients. However, with Brindley stimulation, the electrical stimulation alone does not typically lead to ejaculation.

As such, many typical systems for treating sexual dysfunction do not work in all patients. Many other problems and disadvantages of the prior art will become apparent to one skilled in art after comparing such prior art with the present invention as described herein.

### BRIEF SUMMARY OF THE INVENTION

Aspects of the present invention are found in a method for treating sexual dysfunction. The method includes stimulation of sacral nerve roots near or about the sacrum with a set of electrodes. The electrodes may be placed within the sacrum by percutaneously inserting a lead in the epidural space and retrogradely advancing it caudally towards the desired roots. In addition, paddle type electrodes could be used by surgically placing them within the sacrum so that they lye directly over the desired roots immediately before they exit the foramen. The electrodes may also be inserted along the root as it emerges from the sacrum. Alternately, the electrodes may be placed transverse to the sacrum. In a further embodiment, the electrodes may take the form of cuffs placed on the sacral nerve root or spirals of electrodes placed around the sacral nerve root. The electrodes will most likely be placed on or about the third sacral nerve root. However, they may be placed on any sacral nerve root from one to five.

Another aspect of the invention is found in stimulating the electrodes and thus the sacral nerve root with an implantable stimulator. The implantable stimulator may be activated through the use of an external electromagnetic signal. This signal may emanate from a radio transmitter or a magnet.

Further aspects of the invention are found in a system for performing the method above. The system includes a stimulation generator, an external activation device, and one or more electrodes. The external activation device may be a magnetic device or radio-frequency device. The one or more electrodes may take the form of lamitrodes, percutaneous leads, cylindrical leads, cuffs, or spiraled electrodes.

Additional aspects of the invention may be found in a method for treatment of sexual dysfunction. The method may include surgically implanting an implantable nerve stimulator with electrodes extending in or about the sacral nerve roots. The sacral nerve roots include the S2 or S3 nerve roots, among others. The method may include testing the nerve stimulator, adjusting the location of the electrodes or the stimulation settings of the implantable nerve stimulator to achieve genital stimulations, and completing the surgery.

As such, a system and method for treating sexual dysfunction is described. Other aspects, advantages and novel features of the present invention will become apparent from the detailed description of the invention when considered in conjunction with the accompanying drawings.

### BRIEF DESCRIPTION OF THE DRAWINGS

For a more complete understanding of the present invention and advantages thereof, reference is now made to the following descriptions taken in conjunction with the accompanying drawings in which like reference numbers indicate like features and wherein:

FIG. 1 is a schematic depicting the location of the spinal cord relative to the vertebrae;

FIG. 2 is a schematic diagram depicting dermatomes associated with spinal cord segments;

FIGS. 3A and 3B are schematics depicting the sacrum and associated nerve roots;

FIG. 4 is a schematic diagram depicting exemplary embodiments of electrode placement about the sacrum;

FIG. 5 is a schematic diagram depicting an implantable nerve stimulator, according to the invention;

FIG. 6 is a schematic diagram depicting an exemplary embodiment of an implantable stimulator and nerve cuff;

FIG. 7 is a schematic diagram depicting an electrode spiraled around a sacral nerve root; and

FIGS. 8 and 9 are block flow diagrams depicting an exemplary method for treating sexual dysfunction.

### DETAILED DESCRIPTION OF THE INVENTION

The sacral roots, in particular S2 and S3, have dermatomes associated with the genital region and the interior leg region. Stimulation of the sacral nerve roots in or about the sacrum provides a reliable and immobilized stimulation point. While nerve patterns may vary between patients, it is likely that proper stimulation of S2 or S3 leads to genital stimulation and orgasm.

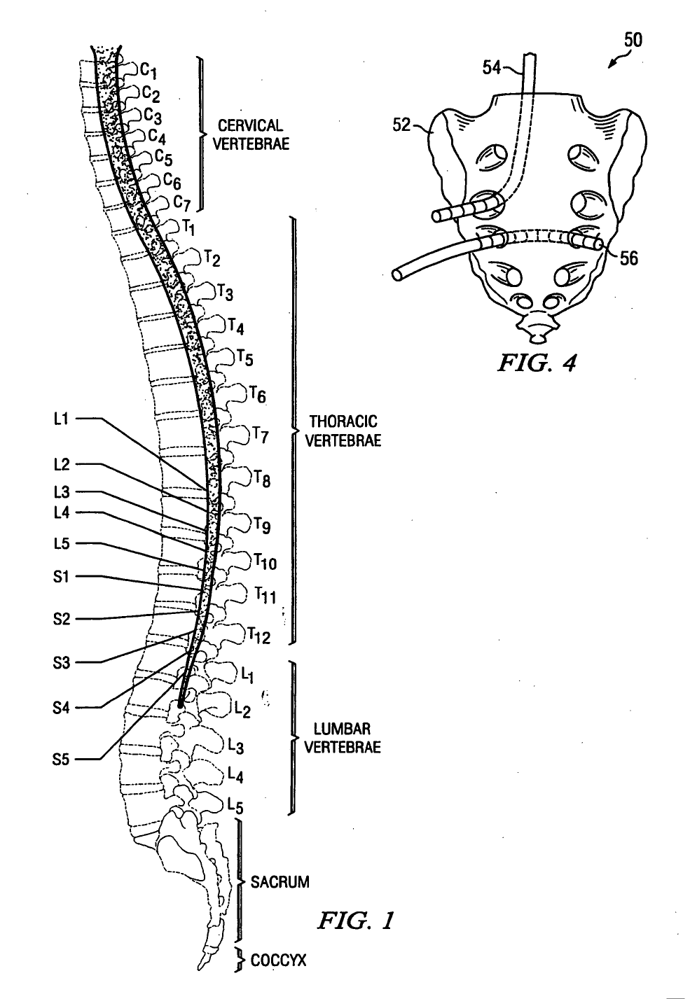

FIG. 1 depicts the spinal cord and the nerve roots extending from it in relation to the vertebrae. As can be seen, the spinal cord terminates in or around the L1 or L2 lumbar vertebrae. In this small termination region between about the T11 to L3 vertebrae, the spinal cord extends a large number of roots including lumbar, sacrum and coccyx roots, about eleven pair all together.

The spinal cord is not fixed in place and as such floats and changes position with changes in posture and body position. Stimulation of a selected region near the end of the spinal cord is difficult because the relative location of the spinal cord changes and the number of nerve roots extending from that region make it difficult to pinpoint the exact nerve bundle desired.

However, the nerve roots can be seen to extend through the vertebra and out their respected foramen. For example, the sacral nerve roots, 1-5, branch from the spinal cord near the L1 vertebral region and emerge from the spinal column in or about the sacrum.

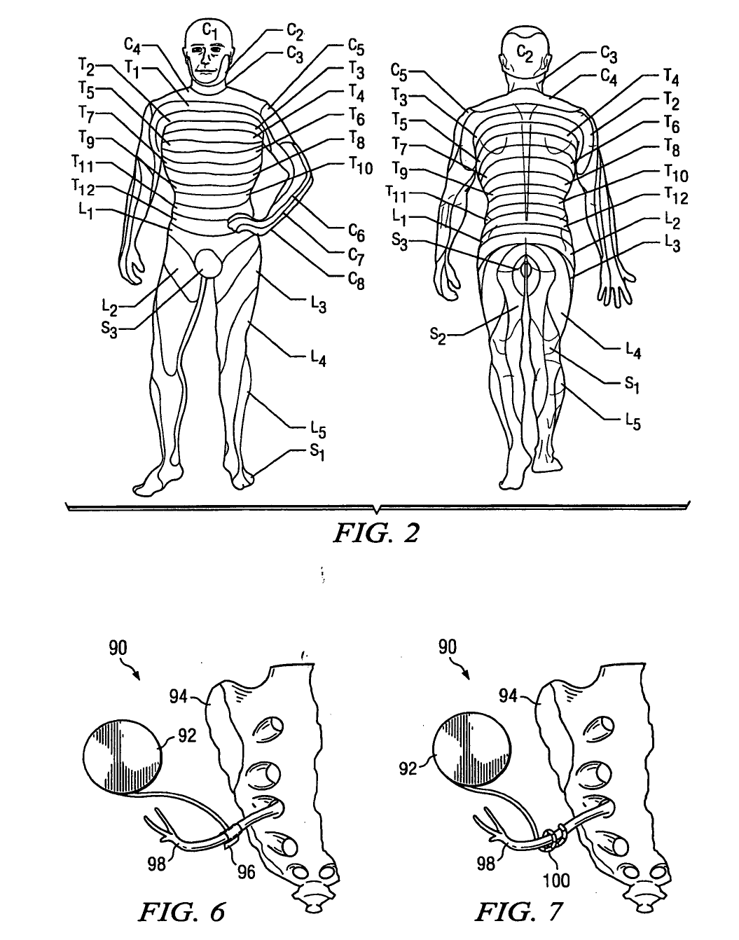

FIG. 2 is a schematic diagram depicting the dermatomes associated with the regions of the spinal cord and the associated nerve roots. Typically, the S2 sacral nerve root is associated with the hind and inner legs and the S3 sacral nerve root is associated with the genital and evacuatory regions. By selectively stimulating the S3 and/or S2 sacral nerve root, genital stimulation may be achieved. However, the required stimulation pattern and selected nerve root may vary between patients.

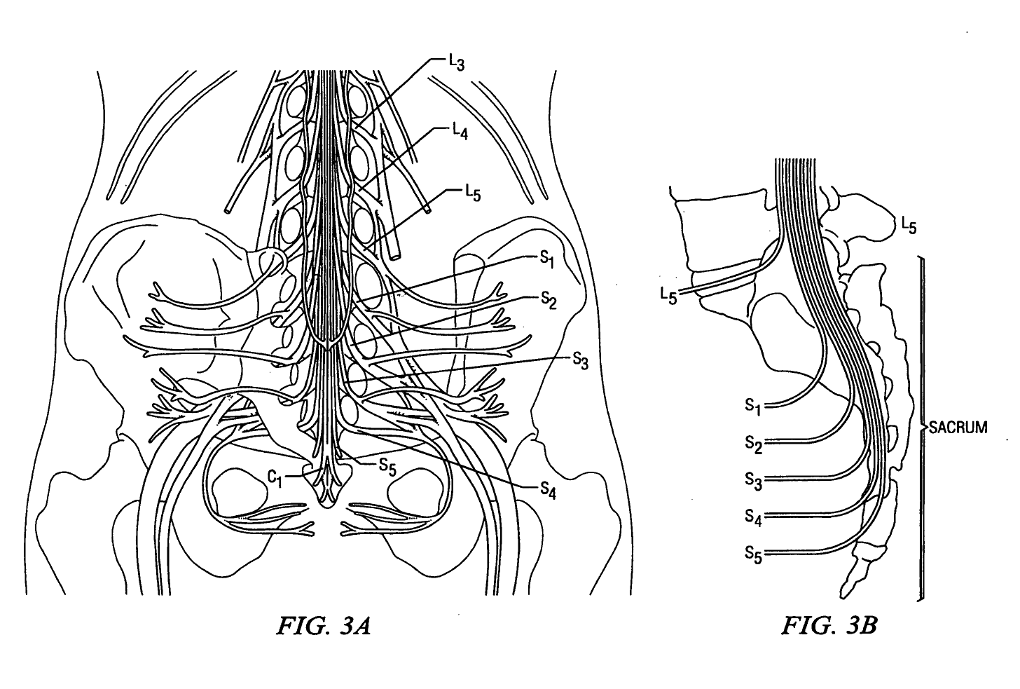

FIG. 3A and FIG. 3B depict the sacrum and the nerve roots extending therefrom. FIG. 3A depicts the front side of the sacrum and FIG. 3B depicts a side view of the sacrum. By selectively stimulating regions of the nerve bundles extending from the sacral nerve roots, genital stimulation may be achieved.

FIG. 4 depicts possible electrode placement for stimulation of the sacral nerve roots. The sacrum 52 is located below the lumbar region of the spine. As seen, the electrodes may be placed along the spinal foramen and emerge with the sacral nerve root. For example, lead 54 is shown extending along the spinal foramen emerging with the second sacral nerve root and terminating in electrodes in and about the region where the sacral nerve root emerges. By selectively programming the electrode stimulation settings, the effect of the stimulation may be varied.

Alternately, the electrode may be placed transverse to the sacrum. As seen with lead 56, the lead may be inserted through the foramen associated with a sacral nerve root and extend through the spinal foramen to an opposite foramen. In this case, a lead with multiple electrodes placed or located where the sacral nerve root emerges may permit stimulation of a desired nerve set. Other exemplary embodiments include placing more than one lead about the sacral nerve root, percutaneous leads, Lamitrodes®, nerve cuffs, and spiraled leads, among others. However, various embodiments of electrode placement and lead design may be envisaged. The placement may also use such methods as described in Feler et. al (U.S. Pat. No. 6,002,964), which is included herein in its entirety be reference. Furthermore, positioning and preferred procedure may vary by patient.

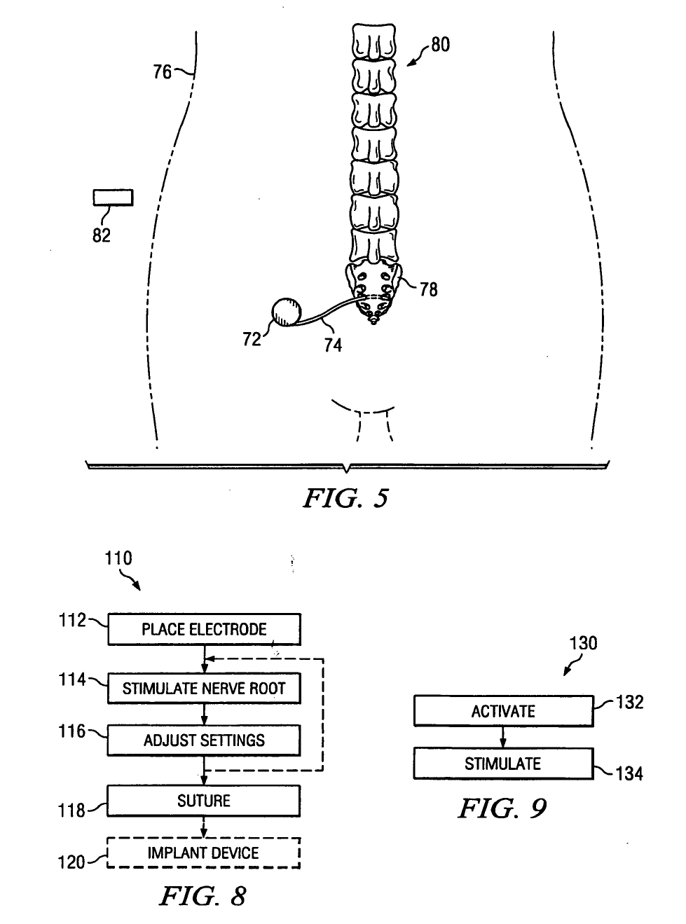

FIG. 5 is a schematic diagram depicting an implantable stimulation device 72. From the implantable stimulation device extends a lead 74. The lead terminates in electrodes placed in or about the sacrum and associated with the sacral nerve roots of a patient 76. The implantable stimulation device 72 may take various forms. These forms may include those that are programmable to deliver various patterns of stimulation pulses to various sets of electrodes. Alternately, the implantable stimulation device 72 may deliver a single pattern of pulses upon activation.

The implantable stimulation device 72 may be activated with an external device 82. This external device may take the form of a magnetic device. Alternately, the external device 82 may take the form of a radio transmitter. In this form, the radio transmitter may permit additional programming to the implantable stimulator 72.

FIG. 6 depicts a further exemplary embodiment of an implantable stimulator. In this case, the implantable stimulator 92 is coupled with an electrode cuff 96. The electrode cuff is wrapped around a sacral nerve 98 as it emerges from the sacrum 94.

FIG. 7 depicts an implantable stimulator 92 attached to one or more leads 100 that terminate in a spiraling set of electrodes surrounding a sacral nerve 98 as it emerges from the sacrum 94. However, various embodiments of the implantable stimulator and electrode configurations may be envisaged.

An alternate embodiment includes using a single electrode or a set of electrodes with a common polarity or a uni-polar configuration. In this embodiment, the stimulation device acts as an electrode with opposite charge to the electrode or electrodes placed in proximity to the sacral nerve root.

However, the electrodes may be placed in various locations proximate to the sacrum, including the epidural space, in the sacrum, in the foramen, and outside the sacrum, among others. The placement may use such methods as described in Feler et. al (U.S. Pat. No. 6,002,964). Various electrode types, locations and arrangements may be envisaged.

FIG. 8 depicts an exemplary method for treating sexual dysfunction. In the method 110, electrodes are placed on or about the sacral root in or around the sacrum. This placement may include the use of one or more leads and may use various configurations of leads and electrodes.

As seen in block 114, the nerve roots may be stimulated to determine their effect. In the event that the desired effect is not achieved, adjustments may be made to the electrode location and stimulation settings as seen in a block 116. This adjustment may include repositioning the electrodes, selection of polarity for various sets of electrodes, and providing settings for a stimulation pulse pattern, among others. The polarity of the electrodes may include negative, positive and neutral. The nerve may be subsequently stimulated and the process repeated until a desired stimulation is achieved.

Stimulation parameters include stimulation amplitude, stimulation pulse width, and stimulation frequency. The stimulation waveform may include a square wave, a passively charge-balanced biphasic wave, an actively charge-balanced biphasic wave. Waveforms may have either an initial anodal or cathodal signal followed by the opposite pulse. Another type of waveform includes two biphasic pulses delivered at a set distance apart (doublette).

As seen in a block 118, the leads may be sutured in place. In addition, a loop of the leads may be sutured in the region to allow for patient movement and to prevent relocation of the electrodes.

The leads may be attached to an implantable stimulation device and this device implanted as seen in block 120. The implantable stimulation device may generate a pattern of stimulation pulses upon activation. These stimulation pulses may have the characteristics described above.

However, these steps may occur in various orders. The method may or may not include all of these steps.

FIG. 9 depicts an exemplary method for using the implanted system. A patient activates the implantable stimulation device as seen in block 132. The activation may use an activation device such as a magnet or radio frequency signal generator. However, several methods may be envisaged.

Block 134 depicts the implantable stimulation device generating a stimulation pulse pattern in the electrodes. This pulse pattern stimulates the selected sacral nerve roots about the sacrum. This stimulation induces sensations and reactions in the genital region and leads to orgasm.

As such, a system for treating sexual dysfunction is described. In view of the above detailed description of the present invention and associated drawings, other modifications and variations will now become apparent to those skilled in the art. It should also be apparent that such other modifications and variations may be effected without departing from the spirit and scope of the present invention as set forth in the claims which follow.

1. A system for treating sexual dysfunction, the system comprising:
at least one electrode located at a sacral nerve root of a body, the at least one electrode located near a sacrum of the body;
a simulation source to provide stimulation to the at least one electrode the stimulation source, generating, upon activation, a stimulation pulse pattern in the at least one electrode effective for genital stimulation; and
an activation device located external to the body, the activation device operable to activate the implantable stimulation pulse generator.

2. The system of claim 1 wherein the activation device comprises a magnet.

3. The system of claim 1 wherein the activation device comprises a radio frequency signal generator.

4. The system of claim 1 wherein the genital stimulation induces orgasm.

5. The system of claim 1 wherein said at least one electrode is disposed about the sacral nerve root.

6. The system of claim 1 wherein said at least one electrode comprises a spiraled lead.

7. The system of claim 1 wherein said at least one electrode comprises a nerve cuff.

8. A system for treating sexual dysfunction, the system comprising:
a lead having at least one electrode thereon, the at least one electrode located at a sacral nerve root of a body; and
a stimulation source to provide stimulation to the at least one electrode located internal to the body, the stimulation source generating, upon activation, a stimulation pulse pattern in the at least one electrode effective for genital stimulation.

9. The system of claim 8 wherein the at least one electrode is disposed in a sacrum of the body.

10. The system of claim 8 wherein the lead is inserted through a foramen associated with the sacral nerve root.

11. The system of claim 8 wherein the lead emerges through a foramen associated with the sacral nerve root.

12. The system of claim 8 wherein said at least one electrode comprises a plurality of electrodes.

13. The system of claim 8 wherein said at least one electrode is provided in a uni-polar configuration.

14. The system of claim 8 further comprising:
an activation device located external to the body, the activation device operable to activate the implantable stimulation pulse generator.

15. A system for treating sexual dysfunction, the system comprising:
at least one electrode located at a sacral nerve root of a body;
an implantable stimulation pulse generator coupled to the at least one electrode and located internal to the body, the implantable stimulation pulse generator generating, upon activation, a stimulation pulse pattern in the at least one electrode effective for genital stimulation; and
an activation device located external to the body, the activation device operable to activate the implantable stimulation pulse generator.

16. The system of claim 15 wherein the at least one electrode is located in a sacrum of the body.

17. The system of claim 15 wherein the at least one electrode is located in the epidural space of the body.

18. The system of claim 15 wherein the at least one electrode is located in a foramen of a sacrum of the body.

19. The system of claim 15 wherein the at least one electrode comprises a spiraled lead.

20. The system of claim 15 wherein the at least one electrode comprises a nerve cuff.
